(19) United States
(12) Patent Application Publication
(10) Pub. No.: US 2024/0261391 A1
(43) Pub. Date: Aug. 8, 2024

(54) TLR7 AGONIST CONJUGATED PEPTIDE-BASED NOVEL CORONAVIRUS NANOEMULSION VACCINE AND PREPARATION THEREOF

(71) Applicants: SHANGHAI INSTITUTE OF MATERIA MEDICA, CHINESE ACADEMY OF SCIENCES, Shanghai (CN); SHENZHEN UNIVERSITY, Guangdong (CN)

(72) Inventors: Xinxin ZHANG, Shanghai (CN); Likun GONG, Shanghai (CN); Jin REN, Shanghai (CN); Guangyi JIN, Guangdong (CN); Wei HUANG, Shanghai (CN); Yong GAN, Shanghai (CN); Jianhua SUN, Shanghai (CN); Tingting LIU, Shanghai (CN); Yiru LONG, Shanghai (CN); Yunqiu MIAO, Shanghai (CN); Qiuping QIN, Shanghai (CN); Feng TANG, Shanghai (CN); Pan YU, Shanghai (CN); Linmiao GUO, Shanghai (CN)

(21) Appl. No.: 18/565,013

(22) PCT Filed: May 30, 2022
(86) PCT No.: PCT/CN2022/096041

§ 371 Date: Nov. 28, 2023

(30) Foreign Application Priority Data
May 30, 2021 (CN) 202110596120.2

(51) Int. Cl. A61K 39/215; A61K 39/00; A61K 47/22; C12N 7/00

(52) U.S. Cl. CPC A61K 39/215; A61K 47/22; C12N 7/00; A61K 2039/55566; C12N 2770/20034

(57) ABSTRACT

The present invention relates to a novel coronavirus vaccine using a TLR7 agonist conjugated peptide as an antigen and an emulsion as an adjuvant. An antigen polypeptide of the conjugated peptide is a polypeptide derived from an S protein of SARS-COV-2, and the adjuvant is an oil-in-water nanoemulsion containing squalene. The conjugated peptide nanoemulsion vaccine preparation of the present invention is thermally stable, and can induce a high level of protective humoral immune response in a cynomolgus monkey, and the neutralizing antibody titer of antiserum after immunization of cynomolgus monkey is high, such that invasion of wild-type strain and mutant novel coronavirus can be blocked. The vaccine of the present invention has a nearly complete protection effect on the upper and lower respiratory tracts of the cynomolgus monkey in a cynomolgus monkey SARS-COV-2 challenge test. The nanoemulsion vaccine of the present invention is fast and convenient to prepare, and can realize large-scale production in a short term for coping with the novel coronavirus outbreak.

### TECHNICAL FIELD

The present invention belongs to the field of biomedical technology, and specifically relates to the preparation of a TLR7 agonist conjugated peptide-based coronavirus SARS-COV-2 nanoemulsion vaccine and its application in preventing infection of coronavirus SARS-COV-2 wild-type and mutant strains.

### BACKGROUND

Severe acute respiratory syndrome coronavirus 2 (SARS-COV-2) is an enveloped positive-sense single-stranded RNA virus, belonging to the family of Coronaviridae, the genera of Betacoronavirus and the species of severe acute respiratory syndrome related-coronavirus. The host of this virus includes mammals and poultry, and it is the pathogen responsible for the Corona Virus Disease 2019 (COVID-19). Up to Apr. 7, 2021, a total of 192 countries and regions worldwide have reported over 132 million confirmed cases, of which over 2.873 million have died and 75.221 million have been cured. Currently, the number is still rapidly increasing. The virus is rapidly spreading while also mutating. In the urgent situation of combating COVID-19, polypeptide vaccines are expected to save time and cost for vaccine development.

Compared with attenuated and inactivated vaccines, viral epitope polypeptide vaccines are more suitable for responding to viral mutations; can meet the requirements of rapid and efficient production, reducing the cost of vaccine production; and do not contain a complete viral structure and have high safety. However, compared to traditional inactivated or live vaccines, modern vaccines composed of genetically engineered recombinant antigens or chemically synthesized polypeptides often have weak immunogenicity. Therefore, immune adjuvants are an important component of synthetic polypeptide vaccine formulations.

Adjuvants in vaccines can effectively enhance the body's immune response to antigens or alter the type of immune response. Currently, various vaccine adjuvants have been developed and approved for marketing, including aluminum adjuvants, emulsions, liposomes, virosomes, etc. Aluminum adjuvants are the most widely used immune adjuvants and can serve as an “antigen library”, slowly releasing antigens, prolonging immune stimulation effects, and promoting the response of macrophages at the injection site. In addition, emulsion-type adjuvants have a long history of research and development, which are currently mainly used as reserve adjuvants for epidemics such as influenza, leishmaniasis and malaria, and have been applied in over 30 countries to date. Unlike the action mechanism of aluminum adjuvants, emulsion-type immune adjuvants mainly indirectly deliver antigens, they can enhance the phagocytosis and pinocytosis of antigenic substances by antigen presenting cells (APCs), stimulate the secretion of factors such as CCL2, CXCL8, CCL3, and CCL4 by monocytes, macrophages, and granulocytes, and promote differentiation of monocyte into DC. Emulsion adjuvants do not directly target DC to enhance antigen uptake, but rather mediate the recruitment and subsequent differentiation of upstream DC precursor cells to exert adjuvant effects. Recent studies have found that emulsion adjuvants can also drain antigens from the injection site to lymph nodes, triggering reactions with immune cells, thereby greatly improving the immune response and producing higher levels of protective antibodies.

Previous studies have shown that aluminum adjuvants, when used together with high-purity small molecule protein antigens, have insufficient ability to stimulate cellular immune responses and are not suitable for the development of polypeptide vaccines. However, the squalene-based oil-in-water nanoemulsion can increase the stability of antigens, which is beneficial for reducing vaccine dose or antigen concentration, and enhancing cellular immune response.

The prescription designs of the approved oil-in-water nanoemulsions of squalene, such as MF-59™ and AS03, are not conducive to the optimal immune response effects of COVID-19 polypeptide antigen. Patent document CN201010247976.0 discloses a submicron emulsion adjuvant containing squalene, polyether, and polyoxyethylene castor oil. However, the polyoxyethylene castor oil has significant toxicity during injection and is prone to allergic reactions, toxic kidney damage, neurotoxicity, and cardiovascular toxicity, etc. Patent document CN200910193930.2 discloses an oil-in-water compound vaccine adjuvant, while the composition of propolis thereinis complex, containing multiple unknown components, and the preparation process requires 70% ethanol, which is prone to solvent residue and is only suitable for veterinary vaccines.

Therefore, there is an urgent need to develop safer and more effective novel adjuvants for human use in this field, which can work in conjunction with TLR7 agonist conjugated peptides. Fast and simple preparation is also required to achieve large-scale production in the short term in response to sudden epidemics.

### SUMMARY OF THE INVENTION

The purpose of the present invention is to provide a TLR7 agonist conjugated peptide-based coronavirus SARS-COV-2 nanoemulsion vaccine and a preparation method thereof.

In the first aspect of the present invention, it provides a coronavirus SARS-COV-2 nanoemulsion vaccine formulation, which comprises:
(a) a coronavirus SARS-COV-2 vaccine polypeptide, comprising an antigen polypeptide and a TLR7 agonist optionally conjugated to the antigen polypeptide;
(b) an adjuvant, which is a squalene-based oil-in-water nanoemulsion; and
(c) a pharmaceutically acceptable carrier, excipient or diluent.

In another preferred embodiment, the adjuvant comprises the following components: 1-15% (w/w) squalene, 0-15% (w/w) α-tocopherol, 0.1-10.0% (w/w) emulsifier, and 0.005-10% (w/w) block copolymer, calculated by the total weight of the formulation.

In another preferred embodiment, the adjuvant further comprises an aqueous medium.

In another preferred embodiment, the adjuvant comprises an oil phase portion and an aqueous phase portion.

In another preferred embodiment, the oil phase portion of the adjuvant comprises: 1-15% (w/w) squalene, 0-15% (w/w) α-tocopherol, and 0.1-10.0% (w/w) emulsifier; and the aqueous phase portion of the adjuvant comprises: 0.005-10% (w/w) block copolymer and an aqueous medium, calculated by the total weight of the formulation.

In another preferred embodiment, the adjuvant comprises 1-5% (w/w) squalene, preferably 2-2.5% (w/w) squalene.

In another preferred embodiment, the adjuvant comprises 0-5% (w/w) α-tocopherol, preferably 2.5-4% (w/w) α-tocopherol.

In another preferred embodiment, the adjuvant comprises 1-5% (w/w) emulsifier, preferably 1-2% (w/w) emulsifier.

In another preferred embodiment, the adjuvant comprises 0.01-5% (w/w) block copolymer, preferably 0.01-0.5% (w/w) block copolymer.

In another preferred embodiment, the squalene is derived from shark liver (especially from the Centrophorus uyato, Deania calcea, southern lanternshark (Etmopterus granulosus), etc.), olive oil, palm oil, wheat germ oil, and/or yeast, etc.

In another preferred embodiment, the emulsifier is selected from the group consisting of: phospholipids, polysorbates, sucrose esters, citric acid fatty acid glycerides, fatty acid glycerides, fatty acid sorbitans, cyclodextrins, polyoxyethylene fatty acid esters, polyoxyethylene fatty alcohol ethers, polyethylene glycols, chitins, chitosans, cholic acid and salts thereof, and a combination thereof.

In another preferred embodiment, the emulsifier is selected from the group consisting of: phospholipids, polysorbates, and a combination thereof.

In another preferred embodiment, the emulsifer comprises polysorbate 80.

In another preferred embodiment, the emulsifer comprises a combination of polysorbate 80 and phospholipids.

In another preferred embodiment, the block copolymer is a medical block copolymer.

In another preferred embodiment, the number-average molecular weight or weight-average molecular weight of the block copolymer is 300-200000, preferably 500-100000.

In another preferred embodiment, the block copolymer is selected from the group consisting of: methoxy polyethylene-glycol polycaprolactone, methoxy polyethylene glycol polylactic acid-hydroxyacetic acid, polylactic acid hydroxyacetic acid-polyethylene imine, polylactic acid-polyethylene glycol, polyphosphoester diblock copolymer, polyoxyethylene polyoxypropylene ether block copolymer, polyethylene oxide-polypropylene oxide-polyethylene oxide triblock copolymer, and a combination thereof.

In another preferred embodiment, the block copolymer is selected from the group consisting of: methoxy polyethylene-glycol polycaprolactone (mPEG-PCL, PEG: 350, 550, 750, 1000, 3400, 5000, 10000, 20000; PCL: 2000, 5000), methoxy polyethylene glycol polylactic acid-hydroxyacetic acid (mPEG-PLGA, PEG: 1000, 2000, 3400, 5000, 10000, 20000; PLGA: 1000, 2000, 5000, 10000, 15000, 20000, 40000), polylactic acid hydroxyacetic acid-polyethylene imine (PLGA-PEI, PLGA: 1000, 2000, 5000, 10000, 15000, 20000, 40000; PEI: 600, 1800, 10000, 70000), polylactic acid-polyethylene glycol (PLA-PEG, PLA: 2000, 5000; PEG: 1000, 2000, 3400, 5000, 10000, 20000), polyphosphoester diblock copolymer (PCL-b-PHEP, PEG: 1000, 2000, 3400, 5000, 10000, 20000), polyoxyethylene polyoxypropylene ether block copolymer (Poloxamer 188, etc.), polyethylene oxide-polypropylene oxide-polyethylene oxide triblock copolymer (PEO-PPO-PEO), and a combination thereof.

In another preferred embodiment, the block copolymer comprises methoxy polyethylene-glycol polycaprolactone copolymer.

In another preferred embodiment, the aqueous medium is selected from the group consisting of: physiological saline, sterilized water, buffered saline, glucose solution, cyclodextrin solution, and a combination thereof.

In another preferred embodiment, the adjuvant further contains an isotonic regulator, with a content of 0.1-8% (w/w).

In another preferred embodiment, the adjuvant further contains a preservative, and the content of the preservative does not exceed 0.1% (w/w).

In another preferred embodiment, the adjuvant has one or more of the following characteristics:
(1) the adjuvant is co-located with the vaccine polypeptide at the injection site, inducing transient cytokine and chemokine responses, and increasing the recruitment of innate immune cells from the bloodstream to the injection site;
(2) the adjuvant enhances the recruitment of innate immune cells at local draining lymph nodes.

In another preferred embodiment, the pharmaceutically acceptable carrier, excipient, or diluent is a physiologically acceptable buffer such as phosphate or citrate.

In another preferred embodiment, the vaccine polypeptide has a structure of Formula I or comprises an oligomer with a structure of Formula I:

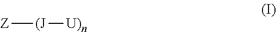

wherein,
Z is an antigen polypeptide with at least one T cell epitope and/or at least one B cell epitope of the novel coronavirus S protein; and the antigen polypeptide has an amino acid sequence derived from the RBM region of the S protein;
each U is independently a TLR7 agonist;
n is 0 or a positive integer;
J is a chemical bond or linker.

In another preferred embodiment, the vaccine polypeptide is selected from the group consisting of: LY54-101, P67-101, and a combination thereof.

In another preferred embodiment, the nanoemulsion vaccine formulation is prepared as an injection.

In another preferred embodiment, the pH of the nanoemulsion vaccine formulation is 6.0-8.0.

In another preferred embodiment, the droplet size in the nanoemulsion vaccine formulation is less than 220 nm, preferably 80-180 nm, and more preferably 100-150 nm.

In another preferred embodiment, the content of the vaccine polypeptide in the nanoemulsion vaccine formulation is 0.1-4 mg/mL.

In another preferred embodiment, the nanoemulsion vaccine formulation has one or more of the following characteristics:
(1) having good stability, and when storing at 4° C. and 40° C. for 1-2 months, the particle size change does not exceed 1%;
(2) the particle size thereof is less than 0.22 μm, which meets the requirements for filtration sterilization.

In the second aspect of the present invention, it provides a method for preparing the vaccine formulation of the first aspect of the present invention, which comprises the following steps:
(S1) providing a vaccine polypeptide;
(S2) mixing the vaccine polypeptide with an adjuvant and pharmaceutically acceptable carrier, excipient, or diluent to prepare the vaccine formulation of the present invention.

Specifically, the method comprises the following steps:
(i) providing a vaccine polypeptide;
(ii) mixing the vaccine polypeptide thoroughly with the adjuvant aqueous phase and/or adjuvant oil phase;
(iii) mixing the adjuvant oil phase with the adjuvant aqueous phase, to form an oil-in-water emulsion through shear stirring or high-pressure homogenization, thereby obtaining the vaccine formulation.

In another preferred embodiment, the vaccine polypeptide is mixed with the adjuvant oil phase.

In another preferred embodiment, the adjuvant oil phase is prepared by the following method: under inert gas protection, stirring and mixing 1-15% (w/w) squalene, 0-15% (w/w) α-tocopherol, and 0.1-10.0% (w/w) emulsifier until a uniform oil phase is formed to obtain an adjuvant oil phase, and each percentage is calculated by the total weight of the formulation; preferably, the inert gas is nitrogen gas.

In another preferred embodiment, the adjuvant aqueous phase is prepared by the following method: 0.01-5% block copolymer is added into an aqueous medium, stirred and dissolved until a uniform aqueous phase is formed, thereby obtaining the adjuvant aqueous phase.

In another preferred embodiment, the method further comprises step (iv), filtrating, sterilizing, and packaging the vaccine formulation obtained in step (iii).

In the third aspect of the present invention, it provides a use of the coronavirus SARS-COV-2 nanoemulsion vaccine formulation, in the manufacture of a medicament for the prevention of coronavirus SARS-COV-2 infection or related diseases.

In another preferred embodiment, the coronavirus SARS-COV-2 comprises a wild-type strain and/or a mutant strain.

In another preferred embodiment, the mutant strain is selected from the group consisting of: B.1.1.7, B.1.617, B.1.351, P.1 and B.1.1.529.

In another preferred embodiment, the coronavirus SARS-COV-2 related disease is selected from the group consisting of: respiratory infection, pneumonia and its complications, and a combination thereof.

In another preferred embodiment, the coronavirus SARS-COV-2 related disease is novel coronavirus pneumonia (COVID-19).

It should be understood that within the scope of the present invention, each technical features of the present invention described above and in the following (such as examples) may be combined with each other to form a new or preferred technical solution, which is not listed herein due to space limitations.

### DESCRIPTION OF THE DRAWINGS

FIG. 1 shows the chemical structure information of LY54-101.

FIG. 2 shows the chemical structure information of P67-101.

FIG. 3 shows the comparison of thermal stability analysis between F2 nanoemulsion and AS03 nanoemulsion of LY54-101.

FIG. 4 shows the fluorescence quantitative results of isolated tissues of heart, liver, spleen, lung, and kidney after 8 hours of intramuscular injection of free conjugated peptide and conjugated peptide nanoemulsion formulation.

FIG. 5 shows the fluorescence imaging quantitative results of inguinal lymph nodes after 8 hours of intramuscular injection of free conjugated peptide and conjugated peptide nanoemulsion formulation.

FIG. 6 shows the serum RBD binding-antibody levels of cynomolgus monkeys immunized with the conjugated peptide nanoemulsion formulation after 35 days.

FIG. 7 shows the serum RBD binding-antibody levels of cynomolgus monkeys immunized with the conjugated peptide nanoemulsion formulation after 70 days.

FIG. 8 shows the serum neutralizing antibody levels of cynomolgus monkeys immunized with the conjugated peptide nanoemulsion formulation after 35 days.

FIG. 9 shows the serum neutralizing antibody levels of cynomolgus monkeys immunized with the conjugated peptide nanoemulsion formulation after 70 days.

FIG. 10 shows that the antiserum of cynomolgus monkeys immunized with the conjugated peptide nanoemulsion formulation can block pseudovirus of novel coronavirus wild-type strain from invading host cells.

FIG. 11 shows that the antiserum of cynomolgus monkeys immunized with the conjugated peptide nanoemulsion formulation can block pseudovirus of novel coronavirus England strain from invading host cells.

FIG. 12 shows that the antiserum of cynomolgus monkeys immunized with the conjugated peptide nanoemulsion formulation can tolerate amino acid mutations on multiple RBDs.

FIG. 13 shows that the antiserum of cynomolgus monkeys immunized with the conjugated peptide nanoemulsion formulation has high neutralizing activity in blocking the invasion of the mutant strain B.1.1.529 pseudovirus into cells.

FIG. 14 shows that the antiserum of cynomolgus monkeys immunized with the conjugated peptide nanoemulsion formulation can block the invasion of euvirus of novel coronavirus into host cells.

FIG. 15 shows the virus load monitoring of nasal and pharyngeal swabs during the virus attack process.

FIG. 16 shows the viral load in the lung tissue of cynomolgus monkeys after the virus attack.

### DETAILED DESCRIPTION

After extensive and in-depth research and extensive screening, the inventors developed a vaccine formulation formula suitable for TLR7 agonist conjugated peptide for the first time, and used the formula to prepare a novel TLR7 agonist conjugated peptide-based coronavirus SARS-COV-2 nanoemulsion vaccine. In the present invention, the TLR7 agonist conjugated peptides are mixed with different formulations of adjuvants to prepare conjugated peptide-adjuvant compositions, and the physicochemical properties of each prepared composition are tested to determine the optimal vaccine formulation formula. In vitro and in vivo experiments have shown that the nanoemulsion vaccine prepared using the preferred formula of the present invention can induce a higher level of humoral immune response after entering the body, stimulate the production of higher titers of neutralizing antibodies, effectively block virus invasion of host cells, thereby having nearly complete protective effects on both the upper and lower respiratory tracts of the body.

Experiments have shown that the antiserum obtained by immunization with the nanoemulsion vaccine of the present invention not only has high neutralizing activity against the wild-type strain of coronavirus SARS-COV-2, but also has considerable neutralizing effect against mutant strains (such as B.1.1.7 and B1.1.529). Therefore, the nanoemulsion vaccine of the present invention can not only be used to prevent the infection of the coronavirus SARS-COV-2 wild-type strain, but also has good preventive effects on the infection of mutant strains.

On this basis, the present invention has been completed.

### Vaccine Polypeptide

The present invention provides a vaccine formulation comprising a coronavirus SARS-COV-2 vaccine polypeptide, wherein the vaccine polypeptide comprises an antigen polypeptide and a TLR7 agonist optionally conjugated to the antigen polypeptide.

In another preferred embodiment, the vaccine polypeptide has a structure of Formula I or comprises an oligomer with a structure of Formula I:

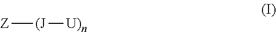

wherein,
Z is an antigen polypeptide with at least one T cell epitope and/or at least one B cell epitope of the coronavirus SARS-COV-2 S protein; and the antigen polypeptide has an amino acid sequence derived from the RBM region of the S protein;
each U is independently a TLR7 agonist;
n is 0 or a positive integer;
J is a chemical bond or linker.

In another preferred embodiment, the vaccine polypeptide can stimulate primates and rodents to produce neutralizing antibodies that block the binding of RBD to ACE2.

In another preferred embodiment, the vaccine polypeptide can stimulate primates to produce cellular immunity and humoral immunity.

In another preferred embodiment, the primates include humans and non-human primates.

In another preferred embodiment, the length of the antigen polypeptide is 8-100 amino acids, preferably 10-80 amino acids.

In another preferred embodiment, the antigen polypeptide has an amino acid sequence derived from the RBD region of the coronavirus SARS-COV-2 S protein.

In another preferred embodiment, the antigen polypeptide has an amino acid sequence derived from the RBM region of the RBD region.

In another preferred embodiment, the RBM region refers to amino acids at positions 438-506 of the coronavirus SARS-COV-2 RBD protein.

In another preferred embodiment, the antigen polypeptide “having an amino acid sequence derived from the RBM region of the RBD protein” means that the amino acid sequence of the antigen polypeptide has homology (or identity) with the RBM region, and the homology is ≥80%, preferably ≥85%, more preferably ≥90%, and most preferably ≥95%.

In another preferred embodiment, the antigen polypeptide is artificially synthesized or recombinant antigen polypeptide.

In another preferred embodiment, the structure of the antigen polypeptide is as shown in Formula II:

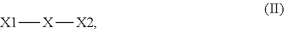

wherein,
(a) X is a core fragment, wherein the sequence of the core fragment is selected from one or more of SEQ ID NOs: 1-12 (see Table A);
(b) each X1 and X2 is independently none, 1, 2, or 3 amino acids, and the total number of amino acids of X1 and X2 is ≤4, preferably 3, 2, or 1, and more preferably 0 or 1; In another preferred embodiment, each X1 and X2 is independently none, K, C, G, L, or A.
(c) “—” represents peptide bond, peptide linker, or other linker (that is, X1 and X and/or X and X2 are connected by peptide bonds, peptide linkers (such as a flexible linker consisting of 1-15 amino acids) or other linkers).

TABLE A
Antigenic peptides

| Peptide ID | Sequence | Length (aa) | SEQ ID No: |
|---|---|---|---|
| LY54 | LFRKSNLKPFERDISTEIYQAGSTPCNG | 54 aa | 1 |
| | VEGFNCYFPLQSYGFQPTNGVGYQPY | | |
| P-67 | GYAWNRKRISNCVADYSVLYNSASFS | 29 aa | 2 |
| | TFK | | |
| P-33 | YNYLYRLFRKSNLKPFERDISTEIY | 25 aa | 3 |
| P-37 | YRLFRKSNLKPFERDISTEIYQAGS | 25 aa | 4 |
| P-41 | KRSFIEDLLFNKVTLADAGFIKQYG | 25 aa | 5 |
| P-67-F1 | CYAWNRKRISN | 11 aa | 6 |
| P-67-F2 | CVADYSVLYNSASFSTFK | 18 aa | 7 |
| P-71 | GVEGFNCYFPLQSYGFQPTNGVGYQP | 28 aa | 8 |
| | YR | | |
| P-73 | FQPTNGVGYQPYRVVVLSFELLHAPAT | 28 aa | 9 |
| | V | | |
| P-79 | ISNCVADYSVLYNSASFSTFKC | 22 aa | 10 |
| P-85 | DYSVLYNSASFSTFKCYGVSPT | 22 aa | 11 |
| P-86 | TEIYQAGSTPCNGVEGENCYFP | 22 aa | 12 |

In another preferred embodiment, the molecule number n of the TLR agonist is 1, 2, 3, 4, 5 or 6; preferably 1, 2, 3 or 4.

In another preferred embodiment, the TLR7 agonist is a small molecule agonist.

In another preferred embodiment, the TLR7 agonist comprises: SZU-101:

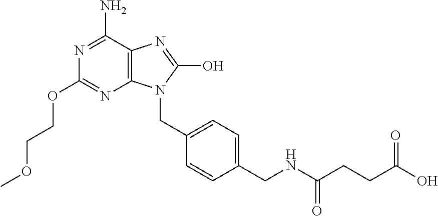 [SMILES: COCCOc1nc(N)c2nc(O)n(Cc3ccc(CNC(=O)CCC(=O)O)cc3)c2n1]

In another preferred embodiment, the TLR7 agonist (such as SZU-101) is attached to the terminal amino group or side chain amino group of the antigen polypeptide.

In another preferred embodiment, the TLR7 agonist (such as SZU-101) is attached to the sulfhydryl group of the antigen polypeptide.

In another preferred embodiment, the SZU-101 is attached to the amino group of the antigen polypeptide and forms the structure shown in S1:

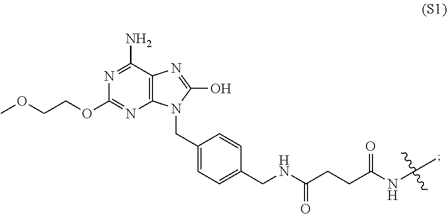 [SMILES: *NC(=O)CCC(=O)NCc1ccc(Cn2c(O)nc3c(N)nc(OCCOC)nc32)cc1]

or
the SZU-101 is attached to the sulfhydryl group of the antigen polypeptide and forms the structure shown in S2:

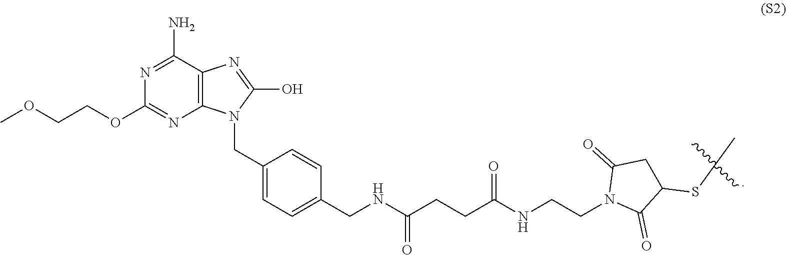 [SMILES: *SC1CC(=O)N(CCNC(=O)CCC(=O)NCc2ccc(Cn3c(O)nc4c(N)nc(OCCOC)nc43)cc2)C1=O]

In another preferred embodiment, the vaccine polypeptide is selected from the group of conjugated peptides consisting of:

(LY54-101, SEQ ID No: 1)
(S1)-LFRK(-S1)SNLK(-S1)PFERDISTEIYQAGSTPCNGVEGFNC
YFPLQSYGFQPTNGVGYQPY

wherein, the SZU-101 is attached to the N-terminal amino group of L and the side chain amino group of K in the amino acid sequence through the S1 structure;

(P67-101, SEQ ID No: 2)
GYAWNRKRISNC(-S2)VADYSVLYNSASFSTFK

wherein, the SZU-101 is attached to the sulfhydryl group of C in the polypeptide through the S2 structure;
and the structures of S1 and S2 are as defined above.

### Squalene

The squalene described in the present invention is a completely metabolizable lipid synthesized by human body through cholesterol synthesis pathway. Squalene mainly comes from shark liver, especially sharks with high levels of squalene, such as the Centrophorus uyato, Deania calcea, and southern lanternshark (Etmopterus granulosus), etc. It can also be extracted from olive oil, palm oil, wheat germ oil, and yeast. Nanoemulsion adjuvants with squalene as the main oil phase can enhance humoral and cellular immune responses, stimulate the maturation of plasma cells, and allow the body to produce sufficient antibodies to fight against viruses. It has been proven in human Phase I to Phase III clinical trials that there are no obvious toxic side effects and it is safe and reliable.

### α-Tocopherol

The α-tocopherol described in the present invention acts as an immune stimulant in emulsions. α-tocopherol is one of the eight isoforms of vitamin E and is the most widely distributed one in nature. Emulsion added with α-tocopherol can increase the uptake of antigens by monocytes, enhance the production of cytokines, and generate higher antibody responses. The α-tocopherol used in the present invention is mainly obtained through synthetic pathways.

### Emulsifier

The emulsifier described in the present invention can be selected from one or more of polysorbate 80 and other polysorbate esters, phospholipids, sucrose esters, citric acid fatty acid glycerides, fatty acid glycerides, fatty acid sorbitans, cyclodextrins, polyoxyethylene fatty acid esters, polyoxyethylene fatty alcohol ethers, polyethylene glycols, chitins, chitosans, cholic acid and salts thereof, etc.

### Block Copolymer

The block copolymer described in the present invention is a biodegradable polymer that can spontaneously form spherical nanoparticles with a core-shell structure with hydrophilic groups facing outward and hydrophobic groups facing inward in water. It can improve drug encapsulation efficiency and biocompatibility, and promote lymph node drainage. In another preferred embodiment, the block copolymer is a medical block copolymer. In another preferred embodiment, the number-average molecular weight or weight-average molecular weight of the block copolymer is 300-200000, preferably 500-100000.

The block copolymer of the present invention comprises one or more of methoxy polyethylene-glycol polycaprolactone (mPEG-PCL, PEG: 350, 550, 750, 1000, 3400, 5000, 10000, 20000; PCL: 2000, 5000), methoxy polyethylene glycol polylactic acid-hydroxyacetic acid (mPEG-PLGA, PEG: 1000, 2000, 3400, 5000, 10000, 20000; PLGA: 1000, 2000, 5000, 10000, 15000, 20000, 40000), polylactic acid hydroxyacetic acid-polyethylene imine (PLGA-PEI, PLGA: 1000, 2000, 5000, 10000, 15000, 20000, 40000; PEI: 600, 1800, 10000, 70000), polylactic acid-polyethylene glycol (PLA-PEG, PLA: 2000, 5000; PEG: 1000, 2000, 3400, 5000, 10000, 20000), polyphosphoester diblock copolymer (PCL-b-PHEP, PEG: 1000, 2000, 3400, 5000, 10000, 20000), polyoxyethylene polyoxypropylene ether block copolymer (Poloxamer 188, etc.), polyethylene oxide-polypropylene oxide-polyethylene oxide triblock copolymer (PEO-PPO-PEO).

### The Nanoemulsion Vaccine of the Present Invention

In the present invention, it provides a coronavirus SARS-COV-2 nanoemulsion vaccine formulation, which comprises:
(a) a coronavirus SARS-COV-2 vaccine polypeptide, comprising an antigen polypeptide and a TLR7 agonist optionally conjugated to the antigen polypeptide;
(b) an adjuvant, which is a squalene-based oil-in-water nanoemulsion; and
(c) a pharmaceutically acceptable carrier, excipient or diluent.

Specifically, the adjuvant comprises the following components: 1-15% (w/w) squalene, 0-15% (w/w) α-tocopherol, 0.1-10.0% (w/w) emulsifier, and 0.005-10% (w/w) block copolymer, calculated by the total weight of the formulation. The adjuvant further comprises an aqueous medium.

The adjuvant used in the vaccine formulation of the present invention comprises an oil phase portion and an aqueous phase portion. Among them, the oil phase portion of the adjuvant comprises: 1-15% (w/w) squalene, 0-15% (w/w) α-tocopherol, and 0.1-10.0% (w/w) emulsifier; and the aqueous phase portion of the adjuvant comprises: 0.005-10% (w/w) block copolymer and an aqueous medium, calculated by the total weight of the formulation.

The novel coronavirus conjugated peptide vaccine of the present invention uses nanoemulsion as adjuvant, wherein the adjuvant is co-located with the antigen at the injection site, inducing transient cytokine and chemokine responses, and increasing the recruitment of innate immune cells from the bloodstream to the injection site. Mainly, monocytes are activated into antigen presenting cells, which carry antigens and migrate to the draining lymph nodes. Furthermore, the adjuvant enhances the recruitment of innate immune cells at local draining lymph nodes. Antigen presenting cells activate immature CD4+T cells, then the activated CD4+T cells interact with antigen specific B cells to induce a large number of memory B cells and plasma cells secreting antibodies.

### Preparation Method

The present invention also provides a method for preparing the vaccine formulation of the first aspect of the present invention, which comprises the following steps:
(S1) providing a vaccine polypeptide;
(S2) mixing the vaccine polypeptide with an adjuvant and pharmaceutically acceptable carrier, excipient, or diluent to prepare the vaccine formulation of the first aspect of the present invention.

Specifically, the method comprises the following steps:
(i) providing a vaccine polypeptide;
(ii) mixing the vaccine polypeptide thoroughly with the adjuvant aqueous phase and/or adjuvant oil phase;
(iii) mixing the adjuvant oil phase with the adjuvant aqueous phase, to form an oil-in-water emulsion through shear stirring or high-pressure homogenization, thereby obtaining the vaccine formulation.

In another preferred embodiment, the vaccine polypeptide is mixed with the adjuvant oil phase.

In another preferred embodiment, the adjuvant oil phase is prepared by the following method: under inert gas protection, stirring and mixing 1-15% (w/w) squalene, 0-15% (w/w) α-tocopherol, and 0.1-10.0% (w/w) emulsifier until a uniform oil phase is formed to obtain an adjuvant oil phase, and each percentage is calculated by the total weight of the formulation; preferably, the inert gas is nitrogen gas.

In another preferred embodiment, the adjuvant aqueous phase is prepared by the following method: 0.01-5% block copolymer is added into an aqueous medium, stirred and dissolved until a uniform aqueous phase is formed, thereby obtaining the adjuvant aqueous phase.

In another preferred embodiment, the method further comprises step (iv), filtrating, sterilizing, and packaging the vaccine formulation obtained in step (iii).

### Application

The coronavirus SARS-COV-2 nanoemulsion vaccine formulation of the present invention can be used for preparing a medicament for the prevention of coronavirus SARS-COV-2 infection or related diseases.

The coronavirus SARS-COV-2 infection comprises infection caused by wild-type strain and/or mutant strains. The coronavirus SARS-COV-2 related diseases include but are not limited to respiratory tract infection, pneumonia and its complications, etc., such as novel coronavirus pneumonia (COVID-19).

Typically, the vaccine formulation of the present invention may be made into injectable formulations for use, such as liquid solutions or suspensions.

The vaccine formulation of the present invention may be made into a unit or multiple dosage form. Each dosage form comprises a predetermined amount of active substance calculated to produce the desired therapeutic effect, and suitable pharmaceutical excipients.

The formulated vaccine formulation may be administered by conventional routes, including (but not limited to): intramuscular, intravenous, intraperitoneal, subcutaneous, intradermal, oral, or topical administration.

When using a (vaccine) composition, a safe and effective amount of the vaccine polypeptide or peptide set of the present invention is administered to a human, wherein the safe and effective amount is usually at least about 1 μg peptide/kg body weight, and in most cases not more than about 8 mg peptide/kg body weight, preferably the dose is about 1 μg-1 mg peptide/kg body weight. Of course, the specific dosage should also consider factors such as the administration route and the patient's health status, which are all within the skill range of a skilled physician.

The beneficial effects of the novel coronavirus vaccine nanoemulsion vaccine injection provided in the present invention include:
(1) The novel coronavirus conjugated peptide vaccine provided in the present invention uses nanoemulsion as adjuvant, which can enhance the recruitment of immune cells at local draining lymph nodes, thereby improving the immune responses and antibodies produced by the polypeptide antigen.
(2) The raw materials used in the nanoemulsion adjuvant of the present invention have high safety, and do not pose safety hazards while exerting auxiliary immune effects.
(3) The nanoemulsion adjuvant and its corresponding nanoemulsion vaccine formulation of the present invention have good stability and can greatly simplify the storage and transportation conditions of the vaccine formulation.
(4) The provided vaccine formula has good compatibility with conjugated peptides, and also has good compatibility in manufacturing, which can be applied to one or more combinations of different conjugated peptides.

The present invention is further explained below in conjunction with specific examples. It should be understood that these examples are only for illustrating the present invention and not intend to limit the scope of the present invention. The conditions of the experimental methods not specifically indicated in the following examples are usually in accordance with conventional conditions as described in Sambrook et al., Molecular Cloning: A Laboratory Manual (New York: Cold Spring Harbor Laboratory Press, 1989), or according to the conditions recommended by the manufacturer. Unless otherwise stated, percentages and parts are calculated by weight.

### Example 1: Preparation of a Nanoemulsion Vaccine Using TLR7 Agonist Conjugated Peptide LY54-101 as an Antigen

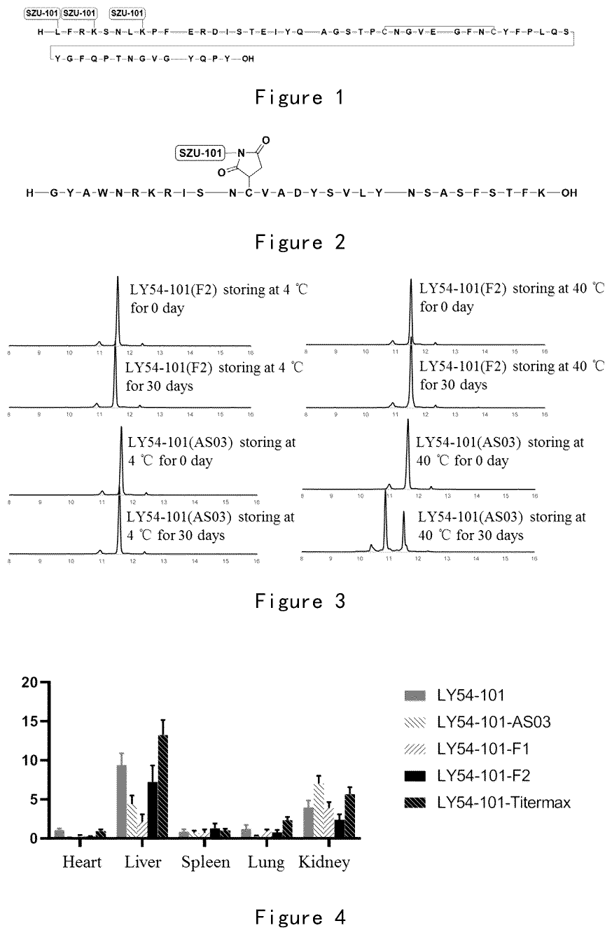

As shown in Table 1 below, a nanoemulsion vaccine using TLR7 agonist conjugated peptide LY54-101 (2 mg/mL) as the antigen was prepared. The structure of LY54-101 was shown in FIG. 1. The preparation method was as follows:
(1) Preparation of oil phase: squalene, α-tocopherol and emulsifier were mixed evenly under nitrogen protection until a uniform oil phase was formed, and it was stored for later use;
(2) Preparation of aqueous phase: in the aqueous phase, 0.01-5% (w/w) block copolymers were added or not to form a uniform aqueous phase for later use;
(3) Polypeptides LY54-101 were fully mixed with the oil phase under nitrogen protection;
(4) The drug-containing oil phase was mixed with the aqueous phase to form an oil-in-water emulsion through shear stirring or high-pressure homogenization;
(5) The nanoemulsion was filterd through 0.22 μm filter membrane, followed by filling, nitrogen filling, and sealing.

TABLE 1
Different Formulas of LY54-101 2 mg/mL

| | | | | | Methoxy | | |
|---|---|---|---|---|---|---|---|
| | | | | Egg yolk | polyethylene | | |
| | | | | lecithin | glycol-polylactic | Add aqueous | |
| | Squalene | Tocopherol | Polysorbate | E80% | acid block | phase to 100% | Particle |
| Formula | % (w/w) | % (w/w) | 80% (w/w) | (w/w) | copolymer % (w/w) | (w/w) | size (nm) |
| 1 | 1 | 2.5 | 1.8 | — | — | PBS buffer | <220 |
| 2 | 2 | 2.5 | 1.8 | — | — | PBS buffer | <220 |
| 3 | 2.5 | 2.5 | 1.8 | — | — | PBS buffer | <220 |
| 4 | 5 | 2.5 | 1.8 | — | — | PBS buffer | <220 |
| 5 | 10 | 2.5 | 1.8 | — | — | PBS buffer | >220 |
| 6 | 15 | 2.5 | 1.8 | — | — | PBS buffer | <220 |
| 7 | 2 | — | 1.8 | — | — | PBS buffer | <220 |
| 8 | 2 | 2.5 | 1.8 | — | — | PBS buffer | <220 |
| 9 | 2 | 4 | 1.8 | — | — | PBS buffer | <220 |
| 10 | 2 | 10 | 1.8 | — | — | PBS buffer | <220 |
| 11 | 2 | 15 | 1.8 | — | — | PBS buffer | <220 |
| 12 | 2 | 5 | 1 | — | — | PBS buffer | >220 |
| 13 | 2 | 2.5 | 2 | — | — | PBS buffer | <220 |
| 14 | 2 | 2.5 | 4 | — | — | PBS buffer | >220 |
| 15 | 2 | 2.5 | 5 | — | — | PBS buffer | >220 |
| 16 | 2 | 2.5 | 10 | — | — | PBS buffer | >220 |
| 17 | 2 | — | 2 | 0.05 | — | PBS buffer | <220 |
| 18 | 2 | 2.5 | 1.8 | — | 0.5 | PBS buffer | <220 |
| 19 | 2.5 | 2.5 | 1.8 | — | 0.01 | PBS buffer | <220 |
| 20 | 2.5 | 2.5 | 1.8 | — | 0.1 | PBS buffer | <220 |
| 21 | 2.5 | 2.5 | 1.8 | — | 0.5 | PBS buffer | <220 |
| 22 | 2.5 | 2.5 | 1.8 | — | 1.0 | PBS buffer | <220 |
| 23 | 2 | 2.5 | 1.8 | — | — | 10% cyclodextrin | <220 |
| | | | | | | solution | |
| 24 | 2 | 2.5 | 1.8 | — | — | Citric acid | <220 |
| | | | | | | buffer solution | |
| 25 | 2 | 2.5 | 1.8 | — | 0.5 | Citric acid | <220 |
| | | | | | | buffer solution | |

According to the particle size in Table 1, it can be seen that in a fixed amount of polypeptide LY54-101, when added with 2.0 to 5.0% squalene, 0.0 to 4.0% α-tocopherol, 0.05% egg yolk lecithin E80, the droplet size of the prepared nanoemulsion was less than 220 nm. The addition of 0.01-1.0% block copolymer had no effect on particle size and could meet the requirements for filtration sterilization, making it the optimal formula.

### Example 2: Preparation of Nanoemulsion Vaccines Using Different Doses and Types of Conjugated Peptides as Antigens

As shown in Table 2, the preparation method for nanoemulsion vaccines using different doses and types of conjugated peptides as antigens was as follows:
(1) Preparation of oil phase: 2.5% (w/w) squalene, 2.5% (w/w) α-tocopherol and 1.8% (w/w) polysorbate 80 were mixed evenly under nitrogen protection until a uniform oil phase was formed, and it was stored for later use;
(2) Preparation of aqueous phase: in the aqueous phase, 0.5% (w/w) methoxy polyethylene glycol-polylactic acid block copolymers were added to form a uniform aqueous phase for later use;
(3) Lipophilic polypeptides were added to the oil phase and water-soluble polypeptides were added to the aqueous phase under nitrogen protection, and they were thoroughly mixed to dissolve;
(4) The polypepide-containing oil phase was mixed with the aqueous phase to form an oil-in-water emulsion through shear stirring or high-pressure homogenization;
(5) The nanoemulsion was filterd through 0.22 μm filter membrane, followed by filling, nitrogen filling, and sealing.

TABLE 2
Different formulas of nanoemulsion vaccines with different
doses and types of conjugated peptides as antigens

| | | | Particle |
|---|---|---|---|
| | Polypeptide | | size |
| Formula | types | Dose (mg/mL) | (nm) |
| 1 | LY54-101 | 1.0 | <220 |
| 2 | LY54-101 | 2.0 | <220 |
| 3 | LY54-101 | 4.0 | <220 |
| 4 | P67-101 | 1.0 | <220 |
| 5 | P67-101 | 2.0 | <220 |
| 6 | P67-101 | 4.0 | Unstable |
| 7 | LY54-101 | 1.0 | <220 |
| | P67-101 | 1.0 | |
| 8 | LY54-101 | 2.0 | <220 |
| | P67-101 | 1.0 | |

According to the particle size in Table 2, the optimized formula can adapt to different types and different doses of conjugated peptides, and the particle size of nanoemulsion was less than 220 nm, which could meet the requirements of filtration sterilization. The structure of conjugated peptide P 67-101 was shown in FIG. 2.

### Example 3: Stability Determination of Nanoemulsion Vaccine

1. Preparation of experimental formulation group:

| | | | | Methoxy | |
|---|---|---|---|---|---|
| | | | | polyethylene | |
| | | | | glycol- | Add |
| | | | Poly- | polylactic | aqueous |
| | | | sorbate | acid block | phase |
| Formu- | Squalene | Tocopherol | 80% | copolymer % | to 100% |
| lation | % (w/w) | % (w/w) | (w/w) | (w/w) | (w/w) |
| AS03 | 2.0 | 2.5 | 1.8 | — | PBS buffer |
| F2 | 2.5 | 2.5 | 1.8 | 0.5 | PBS buffer |

Single conjugated peptide formulation group: AS03 nanoemulsion and F2 nanovaccine emulsion containing 2 mg/mL of LY54-101;

Multiple conjugated peptide formulation group: F2 nanovaccine emulsion containing LY54-101 2 mg/mL+P67-101 1 mg/mL.

### 2. Determination of Polypeptide Content:

The formulations were stored in stability chambers at 4° C. and 40° C., respectively, and samples were taken on days 0, 7, 14, 30, and 60. The particle size was measured using the Malvern Nano-ZS90 dynamic light scattering particle size potential analyzer.

TABLE 3
Stability Results of Single Conjugated Peptide
F2 Nanoemulsion Vaccine Formulation

| Storage | Storage time (days) | | | | |
|---|---|---|---|---|---|
| conditions | 0 | 7 | 15 | 30 | 60 |
| 4° C. (nm) | 116.3 | 116.9 | 117.1 | 116.5 | 116.8 |
| 40° C. (nm) | 116.3 | 117.1 | 116.7 | 117.2 | 116.8 |

TABLE 4
Stability Results of Multiple Conjugated Peptide
F2 Nanoemulsion Vaccine Formulation

| Storage | Storage time (days) | | | | |
|---|---|---|---|---|---|
| conditions | 0 | 7 | 15 | 30 | 60 |
| 4° C. (nm) | 126.5 | 128.3 | 127.6 | 128.1 | 127.4 |
| 40° C. (nm) | 126.5 | 127.9 | 127.4 | 128.3 | 127.8 |

The results show that the nanoemulsion vaccine formulation of the present invention had stable properties and there was no discoloration or layering phenomenon after being stored at 4° C. and 40° C. for 60 days, and the particle size remained basically unchanged.

The effective components of the nanoemulsion vaccine were identified by high-performance liquid chromatography (HPLC). The F2 nanoemulsion vaccine and AS03 nanoemulsion vaccine of the present invention were able to maintain stability of the encapsulated polypeptide components at 4° C. In addition, even after being stored at 40° C. for one month, the effective component LY54-101 of the F2 nanoemulsion vaccine of the present invention remained stable and did not degrade, while the effective component LY54-101 of the nanoemulsion vaccine using AS03 as the control had already degraded (FIG. 3). This indicated that the nanoemulsion of the present invention was more suitable for TLR7 agonist conjugated peptides, which could protect the conjugated peptides from high temperature conditions, thereby maintaining stable properties.

### Example 4: The Effect of Nanoemulsion Vaccine on Lymph Node Drainage

1. Experimental formulation group:

| | | | | Methoxy | |
|---|---|---|---|---|---|
| | | | | polyeth- | |
| | | | | ylene | |
| | | | | glycol- | Add |
| | | | Poly- | polylactic | aqueous |
| | | | sorbate | acid block | phase |
| | Squalene | Tocopherol | 80% | copolymer | to 100% |
| Formula | % (w/w) | % (w/w) | (w/w) | % (w/w) | (w/w) |
| Free | — | — | — | — | PBS buffer |
| conjugated | | | | | |
| peptide | | | | | |
| AS03 | 2.0 | 2.5 | 1.8 | — | PBS buffer |
| F1 | 2.5 | 2.5 | 1.8 | — | PBS buffer |
| F2 | 2.5 | 2.5 | 1.8 | 0.5 | PBS buffer |

The formulations all used LY54-101 connected to Cy5 as the antigen, and the preparation method was the same as described in Example 1. The Titermax group adopted an oil-in-water type TiterMax® adjuvant.

### 2. Experimental Method:

Rats were anesthetized with isoflurane and their left legs were depilated before being placed in a live imaging device for pre administration (0 h) imaging. 100 μL of PBS, free conjugated peptide, AS03, F1, and F2 formulations were injected (respectively) into the inner muscle of the left thigh. After 8 hours of injection, the rats were dissected and their hearts, livers, spleens, lungs, kidneys, and left inguinal lymph nodes were removed for ex vitro tissue fluorescence imaging. The excitation wavelength used was 640 nm and the emission wavelength was 680 nm.

The results indicated (FIG. 4) that the free conjugated peptide group and the Titermax group were mainly distributed in the livers and kidneys, indicating that they were mainly metabolized by the livers and kidneys. The target emulsion groups AS03, F1, and F2 exhibited high fluorescence distribution in the kidneys, indicating that vaccine conjugated peptide emulsion was mainly metabolized by the kidneys.

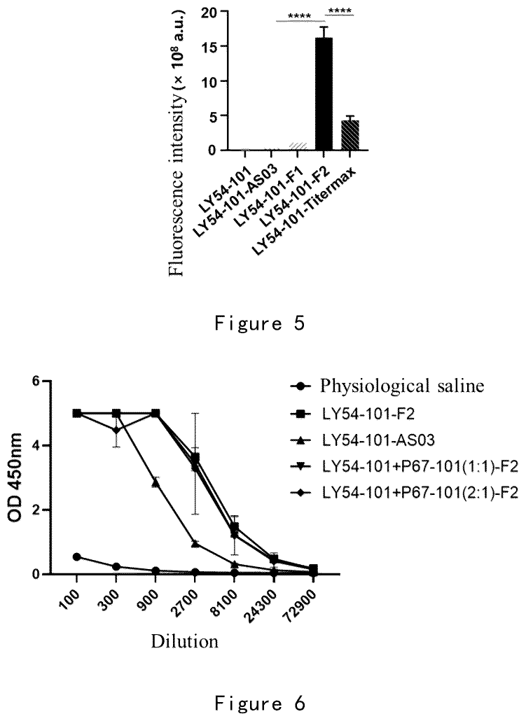

Cy5-LY54-101 had almost no aggregation in lymph nodes, indicating that the immune activation effect of free vaccine conjugated peptides was poor. Emulsions AS03, F1, F2, and Titermax were recruited to varying degrees in lymph nodes, and the fluorescence intensities of AS03 and F1 were both lower than that of the positive control group Titermax (FIG. 5). The F2 emulsion group had the highest fluorescence intensity at the lymph nodes, which was 3.8 times higher than Titemax and 32.9 times higher than the AS03 emulsion group. It is speculated that F2 emulsion may have a better immune activation effect than AS03 emulsion (FIG. 5). This indicates that the vaccine formulation containing block copolymers of the present invention can further promote drug accumulation at the injection site and play a more active and significant role in immune activation.

### Example 5: Immune Response Levels of Nanoemulsion Vaccines

### 1. Animal Immunization Regimen:

Cynomolgus monkeys were divided into groups for intramuscular injection and immunized on days 0, 14, and 28. The regimen was as follows:
Group 1: Blank control group, only giving the same volume of physiological saline.
Group 2: LY54-101 (2 mg, F2);
Group 3: LY54-101 (2 mg, AS03);
Group 4: LY54-101+P67-101 (1:1, 2 mg+2 mg, F2);
Group 5: LY54-101+P67-101 (2:1, 2 mg+1 mg, F2).

### 2. Antibody Level Detection:

To evaluate the level of humoral immune response induced by conjugated peptide nanoemulsion vaccines, it is necessary to detect RBD binding antibodies and neutralizing antibodies in the serum of cynomolgus monkeys after vaccination.

RBD binding antibodies were measured using standard bridged ELISA methods. The specific detection method was as follows: a 96-well ELISA plate was coated with 1 μg/mL of RBD-His at 4° C. for overnight. After washing three times with PBST buffer (containing 0.05% Tween-20 in PBS), the ELISA plate was blocked with 200 μL of 1% BSA solution at 37° C. for 1 hour. After washing, the 96-well plate was added with 100 μL of serum gradient diluent and incubated at 37° C. for 1 hour. After washing, the 96-well plate was added with 100 μL of Protein A-HRP (1:5000), and incubated at 37° C. for one hour and shaken at 650 rpm. After washing, the 96-well plate was incubated with 100 μL of tetramethylbenzidine (TMB) substrate solution at 37° C. and shaken at 650 rpm for 20 minutes. 2M sulfuric acid solution was added to terminate the reaction, and the absorbance value was measured at 450 nm by using the automatic microplate reader SpectraMax.

Neutralizing antibodies were detected using competitive ELISA method. The specific determination method was as follows: the sample and control substance were pre-incubated with HRP-RBD to bind the tested neutralizing antibody to HRP-RBD. Then the mixture was added to the capture plate pre-coated with hACE2 protein. Unbound HRP-RBD and HRP-RBD that bound to non-neutralizing antibodies would be captured on the plate, while the neutralizing antibodies/HRP-RBD complexes in the sample remained in the supernatant and were removed during the washing process. After the washing step, TMB solution was added to turn the color blue. By adding a termination solution, the reaction was quenched and the color turned yellow. This final solution could be read at 450 nanometers in a microplate reader. The absorbance of the sample was inversely proportional to the titer of the neutralizing antibody against SARS-COV-2.

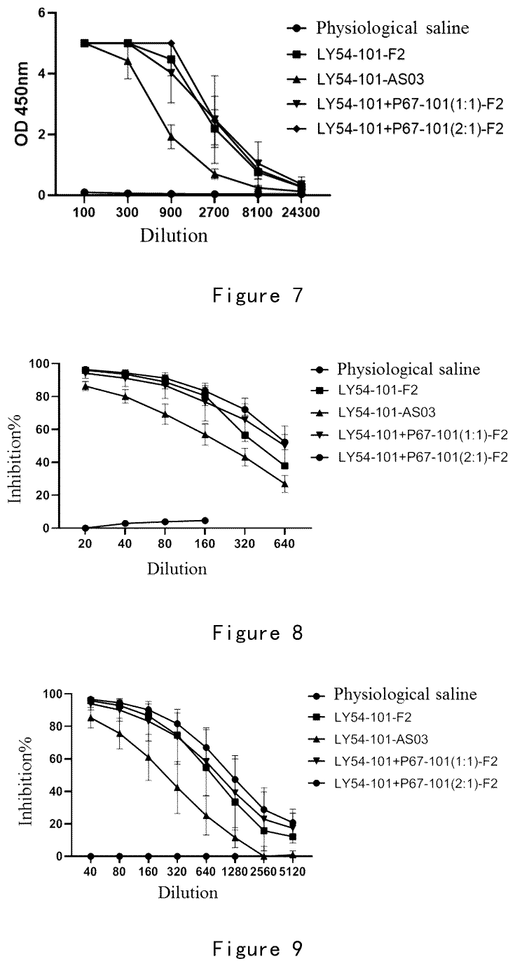

The results indicated (FIGS. 6 and 7) that, based on the serum RBD specific binding antibody levels at the 35th and 70th days after immunization of cynomolgus monkeys, the F2 nanoemulsion vaccine formulation induced significantly higher RBD binding antibody titer than AS03 nanoemulsion, indicating that F2 nanoemulsion was a more suitable vaccine formulation form for the TLR7 agonist conjugated peptide of the present invention. Meanwhile, the LY54-101+P67-101 (2:1, 2 mg+1 mg, F2) group exhibited the highest RBD binding antibody titer, reaching a maximum of 1:72900, indicating that the optimal conjugated peptide combination of the present invention was a 2:1 combination of LY54-101 and P67-101.

Based on the neutralizing antibody levels that blocked the interaction between RBD and ACE2 in the serum of cynomolgus monkeys at the 35th and 70th days after immunization (FIG. 8 and FIG. 9), surprisingly, from 35 to 70 days, the neutralizing antibody levels in the serum continued to increase, reaching a maximum neutralizing antibody titer of 1:5120. Similarly, the vaccine formulation of F2 nanoemulsion induced a significantly higher neutralizing antibody titer than AS03 nanoemulsion, and the LY54-101+P67-101 (2:1, 2 mg+1 mg, F2) group exhibited the highest neutralizing antibody titer.

The above results indicated that the nanoemulsion developed by the present invention had shown better immune effects than AS03 nanoemulsion in the development of conjugated peptide vaccines. Moreover, the conjugated peptide nanoemulsion vaccine of the present invention induced a high-level humoral immune response in the body of cynomolgus monkeys. After immunization, the serum neutralizing antibody levels in cynomolgus monkeys were extremely high and persistent, indicating the ability to block virus invasion.

### Example 6: Nanoemulsion Vaccine Blocking Virus Invasion

In order to further evaluate whether the high-level humoral immune response induced by vaccine immunization of cynomolgus monkeys can block virus invasion of host cells, a pseudovirus testing system and a euvirus testing system were used to evaluate the pseudovirus blocking and neutralizing activity of cynomolgus monkey antiserum.

In the pseudovirus neutralization test, 100 μL of serum samples with different dilutions were mixed with 50 μL of supernatant containing SARS-COV-2 pseudovirus. The mixture was incubated at 37° C. for 1 hour. Then 100 μL of Huh-7/ACE2 cells were added to the mixture of pseudovirus and serum samples, and incubated at 37° C. for another 24 hours. Then, the supernatant was removed and added with 100 μL of luciferase detection solution to each well. After incubation for 2 minutes, the luciferase activity was measured using a microplate photometer.

In the euvirus testing system, the wild-type strain of SARS-COV-2 virus proliferated in VERO E6 cells. Serum samples were thermally inactivated at 56° C. for 30 minutes. Then the twice continuous dilution starting from 1:4 was mixed with an equal volume of virus solution containing 50% tissue culture. The serum-virus mixture was incubated in an environment humidified with 5% CO2 at 37° C. for 1 hour. After incubation, 100 μL of mixture of each dilution was added in duplicate to the cell plate containing a semi-fused VERO E6 monolayer. The plate was incubated at 37° C. for 4 hour. After 4 days of culture, the cytopathic effect (CPE) of each well was recorded under a microscope. The highest serum dilution that could protect more than 50% of cells from CPE was taken as the neutralization titer.

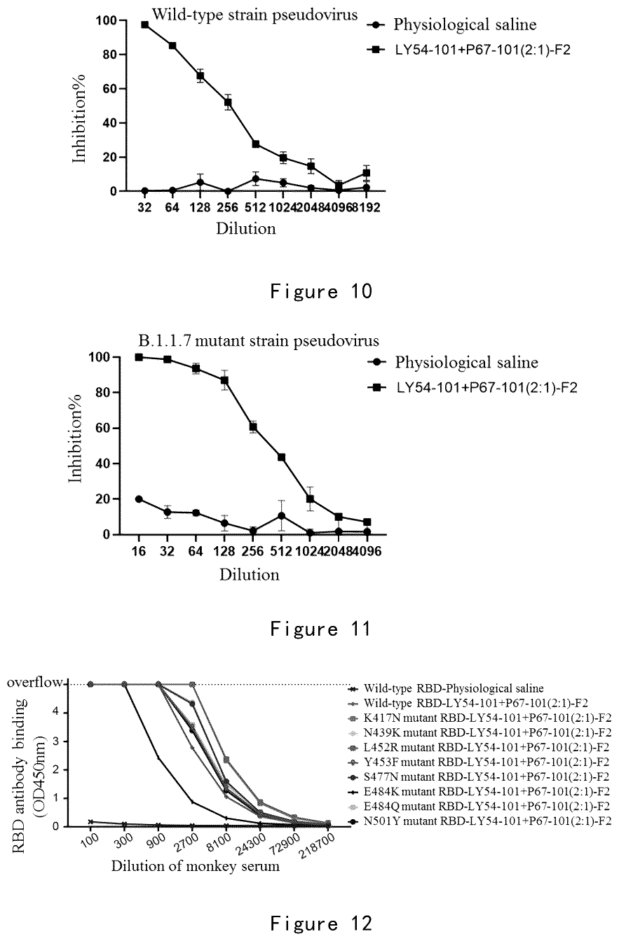

The results of the pseudovirus neutralization experiment (FIG. 10 and FIG. 11) showed that the serum of cynomolgus monkeys immunized with the conjugated peptide nanoemulsion vaccine had a high level of neutralization activity, which could block the invasion of the pseudovirus into host cells, with a titer of up to 1:256 (FIG. 10, wild-type strain).

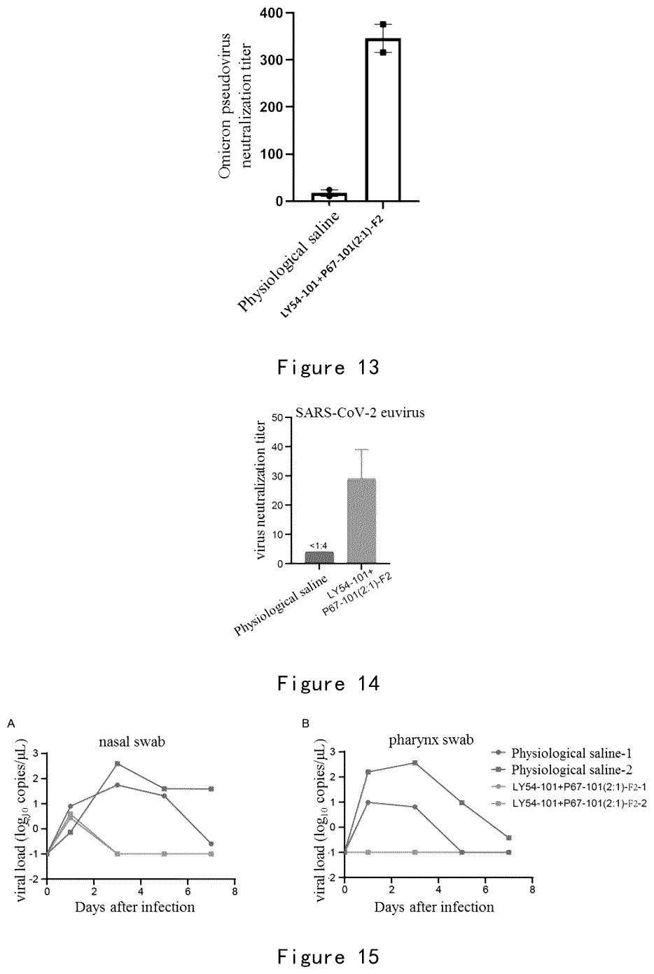

At the same time, the nanoemulsion vaccine of the present invention could effectively protect cells from infection by the mutant strain B.1.1.7, with a titer of up to 1:512 (FIG. 11, mutant strain B.1.1.7). The results showed that the neutralization effect of the nanoemulsion vaccine of the present invention on novel coronavirus mutant strains was equivalent to that of the wild-type strain, so it can also be used to prevent the infection of novel coronavirus mutant strains. In addition, the present invention adopted the RBD binding antibody detection method based on Example 5, replacing the wild-type RBD with multiple single amino acid mutant-RBD proteins of K417N, N439K, L452R, Y453F, S477N, E484K, E484Q, and N501Y for detection. The results showed that after immunizing the cynomolgus monkey with the conjugated peptide nanoemulsion vaccine (on the 35th day), there was no difference in the binding ability of the cynomolgus monkey's antiserum to wild-type RBD when compared to the mutant RBD proteins of K417N, N439K, L452R, Y453F, S477N, E484Q, and N501Y (FIG. 12), indicating that multiple mutations in the RBD proteins did not cause immune escape against the polypeptide vaccine of the present invention. Moreover, the vaccine of the present invention will not undergo immune escape by mutant strains B.1.1.7 (including N501Y mutation) and B.1.617 (including L452R and E484Q mutations). The binding ability of the antiserum of cynomolgus monkey to RBD with E484K mutation decreased three fold (FIG. 12), but still maintained a high antibody titer, indicating that the vaccine of the present invention could still provide good immune protection against mutant strains B.1.351 (including K417N, E484K, and N501Y) and P.1 (including E484K and N501Y). In addition, using the B.1.1.529 (Omicron) pseudovirus model, the antiserum of cynomolgus monkey (62 days after the third immunization) still showed significant neutralizing activity against the Omicron mutant strain, with a neutralizing titer of 1:346 (FIG. 13), indicating that this conjugated peptide nanoemulsion vaccine could protect the body against infection by the Omicron mutant strain.

The results of the envirus neutralization experiment (FIG. 14) showed that the serum of cynomolgus monkeys immunized with the conjugated peptide nanoemulsion vaccine had a high level of neutralization activity, which could block the invasion of the euvirus (wild-type strain) into host cells, with a titer of up to 1:39.

The above results showed that the conjugated peptide nanoemulsion vaccine of the present invention could produce a high level of protective neutralizing antibody after immunizing cynomolgus monkey, and could prevent the infection of novel coronavirus wild-type strain and mutant strains.

### Example 7: Evaluation of the In Vivo Protective Effect of Vaccine Formulations Through Virus Challenge Test

To evaluate the protective effect of the conjugated peptide nanoemulsion vaccine of the present invention in preventing virus infection in vivo, a virus challenge test was conducted on cynomolgus monkeys after 14 days of booster immunization.

The highest viral load in China and abroad (1×10⁷ TCID₅₀) was adopted for virus challenge (commonly 1×10⁶ TCID₅₀), followed by monitoring the viral load of the upper respiratory tract using nasal and pharyngeal swabs on the 1st, 3rd, 5th, and 7th days. On the 7th day, euthanasia was performed on the cynomolgus monkeys, and lung tissues were taken to detect the viral load of each lung lobe. The detection of viral load was carried out through qRT-PCR.

During the virus challenge, the viral load monitoring results of nasal and pharyngeal swabs showed that high viral load was found in the upper respiratory tract of cynomolgus monkeys in the saline group, while only low viral load was detected in the nasal swabs of the cynomolgus monkeys immunized with the conjugated peptide nanoemulsion vaccine of the present invention on the first day after the virus challenge, and no virus RNA was detected in both the nose and pharynx thereafter (FIG. 15).

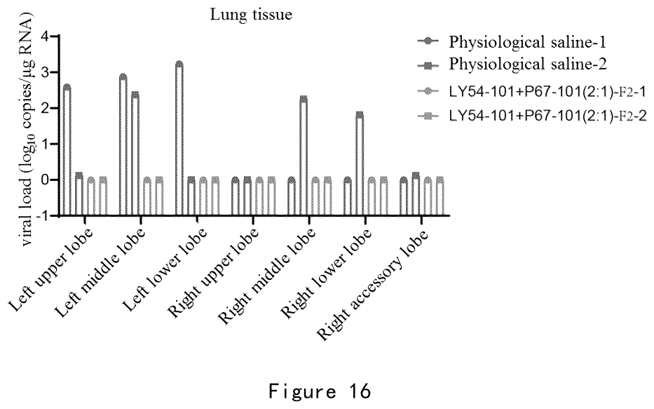

After the end of the virus challenge, the viral load test results of the lung tissues showed that no virus RNA was detected in both the left and right lungs of the cynomolgus monkeys immunized with the conjugated peptide nanoemulsion vaccine of the present invention, while the cynomolgus monkeys in the physiological saline group had high levels of viral load in each lung lobe of the left and right lungs (FIG. 16).

These results demonstrated that the conjugated peptide nanoemulsion vaccine of the present invention had excellent protective effects, and could still prevent the infection of SARS-COV-2 wild-type strain in cynomolgus monkeys at extremely high doses of virus challenge, with nearly complete protective effects on both their upper and lower respiratory tracts.

### Discussion

The present invention develops a safe and effective novel adjuvant for human use based on TLR7 agonist conjugated peptide, which can assist in conjugated peptide to achieve optimal immune effects in vivo. The novel nanoemulsion developed by the present invention exhibits better properties than AS03 nanoemulsion used in clinical practice, such as better thermal stability of the prepared conjugated peptide nanoemulsion formulation, which can withstand high temperature of 40° C. Furthermore, the novel nanoemulsion developed by the present invention has better immune effects, can induce higher levels of neutralizing antibodies against RBD, and can provide efficient protection against SARS-COV-2 wild type and mutant strains, which may be related to its good lymphatic drainage ability.

The TLR7 agonist conjugated peptide nanoemulsion novel coronavirus vaccine of the invention has prominent effects in preventing infection of SARS-COV-2 wild type and mutant strains, can prevent SARS-COV-2 infection, and has nearly complete protective effect on both the upper and lower respiratory tracts of the body, showing clinical values.

All references mentioned in the present application are incorporated by reference herein, as though individually incorporated by reference. In addition, it should be understood that after reading the above teaching content of the present invention, various changes or modifications may be made by those skilled in the art, and these equivalents also fall within the scope as defined by the appended claims of the present application.

1. A coronavirus SARS-COV-2 nanoemulsion vaccine formulation, which comprises:
(a) a coronavirus SARS-COV-2 vaccine polypeptide, comprising an antigen polypeptide and a TLR7 agonist optionally conjugated to the antigen polypeptide;
(b) an adjuvant, which is a squalene-based oil-in-water nanoemulsion; and
(c) a pharmaceutically acceptable carrier, excipient or diluent.

2. The formulation of claim 1, wherein the adjuvant comprises the following components: 1-15% (w/w) squalene, 0-15% (w/w) α-tocopherol, 0.1-10.0% (w/w) emulsifier, and 0.005-10% (w/w) block copolymer, calculated by the total weight of the formulation.

3. The formulation of claim 1, wherein the adjuvant comprises an oil phase portion and a aqueous phase portion, the oil phase portion of the adjuvant comprises: 1-15% (w/w) squalene, 0-15% (w/w) α-tocopherol, and 0.1-10.0% (w/w) emulsifier; and the aqueous phase portion of the adjuvant comprises: 0.005-10% (w/w) block copolymer and an aqueous medium, calculated by the total weight of the formulation.

4. The formulation of any one of claims 1 to 3, wherein the squalene is derived from shark liver, olive oil, palm oil, wheat germ oil, and/or yeast.

5. The formulation of claim 2 or 3, wherein the emulsifier is selected from the group consisting of: phospholipids, polysorbates, sucrose esters, citric acid fatty glycerides, fatty acid glycerides, fatty acid sorbitans, cyclodextrins, polyoxyethylene fatty acid esters, polyoxyethylene polyoxypropylene copolymers, polyoxyethylene fatty alcohol ethers, polyethylene glycols, Poloxamer, chitins, chitosans, cholic acid and salts thereof, and a combination thereof.

6. The formulation of claim 2 or 3, wherein the number-average molecular weight or weight-average molecular weight of the block copolymer is 300-200000, preferably 500-100000.

7. The formulation of claim 2 or 3, wherein the block copolymer is selected from the group consisting of: methoxy polyethylene-glycol polycaprolactone, methoxy polyethylene glycol polylactic acid-hydroxyacetic acid, polylactic acid hydroxyacetic acid-polyethylene imine, polylactic acid-polyethylene glycol, polyphosphoester diblock copolymer, polyoxyethylene polyoxypropylene ether block copolymer, polyethylene oxide-polypropylene oxide-polyethylene oxide triblock copolymer, and a combination thereof.

8. The formulation of claim 3, wherein the aqueous medium is selected from the group consisting of: physiological saline, sterilized water, buffered saline, glucose solution, cyclodextrin solution, and a combination thereof.

9. The formulation of claim 2, wherein the adjuvant further contains an isotonic regulator, with a content of 0.1-8% (w/w).

10. The formulation of claim 1, wherein the vaccine polypeptide has a structure of Formula I or comprises an oligomer with a structure of Formula I: 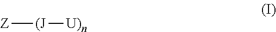
wherein,
Z is an antigen polypeptide with at least one T cell epitope and/or at least one B cell epitope of the novel coronavirus S protein; and the antigen polypeptide has an amino acid sequence derived from the RBM region of the S protein;
each U is independently a TLR7 agonist;
n is 0 or a positive integer;
J is a chemical bond or linker.

11. The formulation of claim 1, wherein the content of the vaccine polypeptide in the nanoemulsion vaccine formulation is 0.1-4 mg/mL.

12. The formulation of claim 1, wherein the nanoemulsion vaccine formulation has one or more of the following characteristics:
(1) having good stability, and when storing at 4° C. and 40° C. for 1-2 months, the nanoemulsion particle size change in the formulation does not exceed 1%;
(2) the nanoemulsion particle size thereof is less than 0.22 μm, which meets the requirements for filtration sterilization.

13. A method for preparing the formulation of claim 1, which comprises the steps of:
(S1) providing a vaccine polypeptide;
(S2) mixing the vaccine polypeptide with an adjuvant and pharmaceutically acceptable carrier, excipient, or diluent to prepare the vaccine formulation of claim 1.

14. Use of the coronavirus SARS-COV-2 nanoemulsion vaccine formulation of any one of claims 1 to 12 in the manufacture of a medicament for the prevention of coronavirus SARS-COV-2 infection or related diseases.

15. The use of claim 14, wherein the coronavirus SARS-COV-2 comprises a wild-type strain and/or a mutant strain, and the mutant strain is selected from the group consisting of: B.1.1.7, B.1.617, B.1.351, P.1, and B.1.1.529.


SEQUENCE LISTING (12 sequences)

SEQ ID NO: 1 - protein, 54 aa, SARS-CoV-2
LFRKSNLKPFERDISTEIYQAGSTPCNGVEGFNCYFPLQSYGFQPTNGVGYQPY

SEQ ID NO: 2 - protein, 29 aa, SARS-CoV-2
GYAWNRKRISNCVADYSVLYNSASFSTFK

SEQ ID NO: 3 - protein, 25 aa, SARS-CoV-2
YNYLYRLFRKSNLKPFERDISTEIY

SEQ ID NO: 4 - protein, 25 aa, SARS-CoV-2
YRLFRKSNLKPFERDISTEIYQAGS

SEQ ID NO: 5 - protein, 25 aa, SARS-CoV-2
KRSFIEDLLFNKVTLADAGFIKQYG

SEQ ID NO: 6 - protein, 11 aa, Artificial sequence
  synthetic peptide
CYAWNRKRISN

SEQ ID NO: 7 - protein, 11 aa, Artificial sequence
  synthetic peptide
CYAWNRKRISN

SEQ ID NO: 8 - protein, 28 aa, SARS-CoV-2
GVEGFNCYFPLQSYGFQPTNGVGYQPYR

SEQ ID NO: 9 - protein, 28 aa, SARS-CoV-2
FQPTNGVGYQPYRVVVLSFELLHAPATV

SEQ ID NO: 10 - protein, 22 aa, SARS-CoV-2
ISNCVADYSVLYNSASFSTFKC

SEQ ID NO: 11 - protein, 22 aa, SARS-CoV-2
DYSVLYNSASFSTFKCYGVSPT

SEQ ID NO: 12 - protein, 22 aa, SARS-CoV-2
TEIYQAGSTPCNGVEGFNCYFP